(19) United States
(12) Patent Application Publication
(10) Pub. No.: US 2005/0100565 A9
(43) Pub. Date: May 12, 2005

(54) Aqueous cosmetic composition comprising porous silica particles and at least one humectant

(76) Inventors: Dhaval Patel, Edison, NJ (US); Carolyn Caes, Mahwah, NJ (US); Gary G. Graves, Morganville, NJ (US)

Correspondence Address: Thomas L. Irving;FINNEGAN, HENDERSON, FARABOW,, GARRETT & DUNNER, L.L.P., 1300 I Street, N.W., Washington, DC, 20005-3315, US

(21) Appl. No.: 10/186,737

(22) Filed: Jul. 2, 2002

(51) Int. Cl. A61K 7/00

(52) U.S. Cl. 424/401

(57) ABSTRACT

An aqueous composition for the skin, comprising porous silica particles having an oil absorption capacity of at least four times by weight their own weight, at least one humectant, and at least 20% water, wherein the porous silica particles and the at least one humectant are present in a combined amount effective to provide moisturization and oil absorption to the skin. Also, a method for providing moisturization and oil absorption to skin by applying the inventive composition to the skin.

The present invention relates to an aqueous composition for skin, wherein the composition contains porous silica particles having an oil absorption capacity of at least four times by weight their own weight and at least one humectant, and at least 20% water, and wherein the porous silica particles and the at least one humectant are present in the composition in a combined amount effective to both moisturize and absorb excess oil from the skin. The invention also relates to methods for providing moisturization and oil absorption to the skin by applying to the skin the compositions of the invention.

Silica shells are known in the art and may, for example, be hollow ellipsoids of silica with a large surface area, high porosity, low bulk density, and high absorption capacity, e.g., up to seven times their own weight. Suitable silica shells are sold, for example, by Kobo Products under the name Silica Shells.

The integrity of the film formed when a composition containing silica shells is applied to the skin may be maintained because the film is not likely to be disrupted, i.e., neither perspiration nor oil are likely to disrupt the film. This allows for a long-lasting “finish” and a non-greasy feeling on the skin. Thus, silica shells may be used in an oil control system.

Silica shells for use in an oil control system may, for example, initially contain at least one volatile material, such as water, which evaporates after application onto a substrate, such as the skin, leaving space available within the shells to absorb oils such as, for example, sebum. However, the silica shells may also wick away moisture from the skin, for example, by absorbing perspiration and releasing it as vapor. In many applications, absorption of oil, if not balanced with a moisturizing effect, results in the skin becoming tight and uncomfortable.

However, the present inventors have discovered that the combination of porous silica particles and at least one humectant in an aqueous composition may both moisturize skin and absorb oil on skin. Accordingly, the present invention, in one embodiment, is drawn to an aqueous composition comprising porous silica particles and at least one humectant in a combined amount effective to provide both moisturization and oil absorption to the skin. The invention is also drawn to methods for providing moisturization and oil absorption to the skin by applying the inventive compositions.

It is to be understood that both the foregoing general description and the following detailed description are exemplary and explanatory only and are not restrictive of the invention as claimed. Reference will now be made in detail to exemplary embodiments of the present invention.

As used herein, “at least one” means one or more and thus includes individual components as well as mixtures/combinations. Further, as used herein, “skin” is meant to comprise facial skin, skin on the body, scalp and lips.

As defined herein, “porous silica particles” refer to silica particles having an oil absorption capacity of at least four times by weight their own weight, such as, for example, at least five times by weight their own weight, at least six times by weight their own weight, and at least seven times by weight their own weight. The porosity of the silica particles ensures that the oil is adsorbed and/or absorbed at at least one location chosen from within the pores of the silica particles, within the interior of hollow silica particles, and between the individual silica particles.

The skilled artisan may determine by routine experimentation the oil absorption of porous silica particles. For example, the oil absorption of silica shells sold by Kobo is tested by weighing a specific amount, e.g., 1 g, of thoroughly mixed and air dried silica shells and placing the shells on a smooth glass plate. A dropping bottle containing linseed oil along with a pipette and rubber bulb are weighed to 0.1 g. Linseed oil is then added gradually and dropwise to the silica shells. After the addition of each drop, the oil and the silica are thoroughly mixed by rubbing with a sharp-edged spatula having a blade ranging from 0.5 inches to 0.75 inches. The test is complete when exactly enough oil has been incorporated with the silica shells to produce a very stiff, putty-like paste that does not break or separate. The dropping bottle containing linseed oil along with a pipette and rubber bulb are then weighed again to 0.1 g. The final weight of the bottle, oil, pipette and rubber bulb is subtracted from the initial weight thereof to determine the weight of oil absorbed by the silica shells. The oil absorption capacity of the silica shells is the ratio of the weight of the oil absorbed over the initial weight of the silica shells, in terms of unit of weight silica shell per unit of weight oil. Accordingly, one of ordinary skill in the art will recognize that the oil absorption of the porous silica particles will depend on the method and the oil used, such as linseed oil (boiled or raw) or mineral oil.

As used herein, the “silica particles” includes silica particles of any color, shape, and size, such as coated and uncoated silica shells, coated and uncoated silica spheres (including microspheres), and coated and uncoated silica particles.

Porous silica particles suitable for use in the present invention is microporous silica particles have an oil absorption capacity of at least four times by weight their own weight. A non-limiting example of porous silica particles suitable for use in the present invention is silica shells having an oil absorption capacity of at least four times by weight their own weight.

As described above, the present invention, in certain embodiments, relates to methods and compositions for providing moisturization and oil absorption to skin comprising porous silica particles, at least one humectant, and at least 20% water, wherein the porous silica particles and the at least one humectant are present in the inventive compositions in a combined amount effective to provide moisturization and oil absorption to the skin. In one embodiment, water is present in the aqueous composition in an amount greater than 20% by weight relative to the total weight of said composition, such as greater than or equal to 30% by weight. In one embodiment, water is present in the aqueous composition in an amount greater than or equal to 40% by weight relative to the total weight of said composition, such as greater than or equal to 50% by weight. In one embodiment, water is present in the aqueous composition in an amount greater than or equal to 60% by weight relative to the total weight of said composition, such as greater than or equal to 70% by weight. In one embodiment, water is present in the aqueous composition in an amount greater than or equal to 80% by weight relative to the total weight of said composition, such as greater than or equal to 90% by weight.

“Oil absorption” is defined herein as reduction or elimination of oil on a substrate, such as skin. Oil absorption includes in its scope “oil control,” i.e., the reduction or elimination of the amount of oil on the skin, as well as what is known in the cosmetic art as “shine control,” i.e., the reduction or elimination of the amount of oil on the skin where there is enough excess oil to cause untreated skin to have a “shiny” appearance.

As previously defined, oil absorption includes oil control. The ability of a composition to provide oil control to skin is evaluated using blotting paper. See Example 1. Further, also as previously defined, oil absorption includes shine control.

The ability of a composition to provide shine control to skin is evaluated by measuring the gloss of skin using a gloss meter. See Example 2.

“Moisturizing” is defined herein as hydrating a substrate, such as the skin, and also retaining moisture in the substrate. Many types of moisturizers are known, such as humectants, which hydrate the skin by stabilizing the water content of a material and, in effect, “holding” water on the skin.

“Humectant,” as used herein, refers to a substance having an affinity for water and a stabilizing action on the water content of material, i.e., promotes water retention. As used herein, “humectant” does not include substances which do not possess both of the above-mentioned characteristics.

The degree of moisturization imparted to skin may be evaluated by measuring the hydrating efficiency of a composition, for example, using a Corneometer which measures the conductivity of the skin. Accordingly, the moisturization of the skin prior to and following application of the composition may be quantitatively measured and compared, for example, with untreated skin and/or a control composition. (See Example 2).

Any porous silica particles having an oil absorption capacity of at least four times by weight their own weight are useful in the practice of the invention. The porous silica particles, for example, may be hollow ellipsoids of porous silica. A non-limiting example of one type of porous silica particles are silica shells. A non-limiting example of one type of silica shells useful in the present invention is that available from Kobo Products. The process for preparation of these shells has been patented by DuPont (see U.S. Pat. Nos. 5,024,826, 5,512,094, and 5,545,250, and WO 94/24677) and licensed to Kobo. Porous silica of this type may be prepared, for example, by coating a calcium carbonate particle with silica. The carbonate is then leached out, leaving only the outer silica coating or shell. Such an outer coating may, for example, range from 0.04 microns to 0.08 microns thick. In one embodiment, the silica shell particles may, for example, average 3 microns in diameter. Due to their morphology and porous nature, the silica shells may have a large surface area (e.g., 100 m²/g) and high oil absorption (e.g., 600 g of 70 cSt mineral oil per 100 g powder). The shells may be thought of as “micron sized silica sponges.”

The oil absorption by the shells may result from absorption at at least one of three locations: within the pores of the shell wall, within the interior of the hollow ellipsoids, and between the individual particles. In one embodiment, the volume of the pores in the shell wall, which may range from 17 Angstroms to 3000 Angstroms, may be 0.2 cc/g, as measured by nitrogen gas adsorption. In another embodiment, the volume of the hollow ellipsoids may be 3 cc/g. Of course, there is no requirement that the silica shells be uniform in size and, in fact, shells of varying sizes may be used in a single formulation.

Another non-limiting example of one type of porous silica particles are spherical porous silica particles. A non-limiting example of one type of spherical porous silica particles useful in the present invention is spherical microporous silica, such as SUNSPHERE, available from AGA Chemicals, Inc. SUNSPHERE is spherical microporous silica (amorphous silicon dioxide) and contains no crystalline silica.

SUNSPHERE having an oil absorption capacity of at least four times by weight their own weight are porous silica particles suitable for use in the present invention. Due to their morphology and porous nature, suitable SUNSPHERE may, for example, have a large surface area (e.g., 700 m²/g), a high pore volume (e.g., 2 mL/g), and a large pore diameter (e.g., 300 Å).

While the porous silica particles may be any color, in one embodiment they are available in the form of a white powder, which gives a white color to formulations but may be transparent when rubbed on the skin and may blend easily into either the water or oil phase, producing a stable emulsion that will not whiten the skin. The porous silica particles may, for example, be used over a large range of processing temperatures, e.g., from 0° C. to 120° C.

The porous silica particles may be uncoated, hydrophobically-coated or hydrophically-coated. However, the porous silica particles of the present invention do not include porous microspheres coated with a perfluorinated oil, a fluorinated silicone oil or a silicone gum. The porous silica particles may be coated with metal oxides such as titanium, iron hydroxide, and iron oxide hydroxide (FeOOH) or with metals such as silver. As used herein, “coated” means has undergone a process of surface-treatment prior to addition to a composition, i.e., surface-treatment is not an in situ process. Like Attorney Docket No.: 05725.0847-00 the uncoated shells, the coated particles may also disperse easily into water or oil. The FeOOH coated particles may have an orange to brown color, depending on the amount of FeOOH used. Iron oxide coated particles may be coated with oxides chosen from yellow, red, and black iron oxides. For example, the iron oxides may be prepared as iron oxide dispersions using iron oxides treated with isopropyl titanium triisostearate in isododecane or isohexadecane as the vehicles. However, the coated particles may, for example, have a somewhat lower oil absorption than the corresponding uncoated particles, depending on the coating. Accordingly, only coated particles having an oil absorption capacity of at least four times by weight their own weight are within the scope of the present invention.

In one embodiment, the porous silica particles are not adsorbed with the at least one humectant, i.e., the porous silica particles are not pretreated, and thereby coated with, the at least one humectant. In another embodiment, the porous silica particles are adsorbed with at least one humectant. In one embodiment, the porous silica particles are not preloaded. In another embodiment, the porous silica particles are preloaded, i.e., filled with a substance, such as water, a polymer, an oil, a humectant, or a moisturizer. However, in the case of preloaded porous silica particles, the inventive compositions further comprise at least one component chosen from water and at least one humectant which is not exclusively comprised within the porous silica particles prior to application to skin. That is, the inventive compositions are not “powder-to-liquid” compositions.

According to the present invention, the porous silica particles and the at least one humectant are present in the composition in a combined amount effective to provide moisturization and oil absorption to the skin. When the inventive composition is on the skin, the porous silica particles may absorb, for example, at least one of water, alcohol, and other organic fluids such as sebum, and mineral oils. One of ordinary skill in the art will recognize that the amount effective to provide moisturization and oil absorption to the skin of the porous silica particles and the at least one humectant will be dependent on, inter alia, the amount of porous silica particles, the amount of the at least one humectant, the oil absorption capacity of the porous silica particles, as well as the presence or absence of any components known in the art to absorb oil, produce or comprise oil. For example, certain pigments may absorb oil.

Routine experiments for determining the combined amount effective to provide moisturization and oil absorption to the skin of porous silica particles and of the at least one humectant which may be useful in the practice of the invention include blot testing, as described in Example 1, which determines the oil control of a composition;

the shine control test, as described in Example 2, which determines whether a composition is capable of controlling shine on the skin where there is enough excess oil to cause untreated skin to have a “shiny” appearance; and the moisturization test, as described in Example 2, which tests the hydrating efficiency of a composition on the skin.

The porous silica particles may, in one embodiment, be present in the inventive composition in an amount ranging from 0.1% to 5% by weight relative to the total weight of the composition, such as from 0.5% to 5% by weight. In another embodiment, the porous silica particles may be present in an amount ranging from 0.6% to 1.2% by weight relative to the total weight of the composition, such as from 1.0% to 1.2% by weight. In a further embodiment, the porous silica particles are present in an amount greater than 0.6% by weight relative to the total weight of the composition. In yet another embodiment, the porous silica particles and the at least one humectant are present in a combined amount effective to provide both moisturization and shine control to the skin for at least eight hours, such as up to eight hours.

Non-limiting examples of the at least one humectant include those listed on pages 1761, 1773 and 1774 of the CTFA International Cosmetic Ingredient Dictionary and Handbook, 8^{th} Ed. (2000). In one embodiment, the at least one humectant may be chosen from glycerin, glycols, sodium hyaluronate, and sodium pyrrolidone carboxylic acid (sodium PCA). In another embodiment, the at least one humectant is glycerin.

The at least one humectant may, for example, be present in the inventive composition in an amount ranging from 0.001 % to 20% by weight relative to the total weight of the composition. In another embodiment, the at least one humectant may be present in an amount ranging from 1% to 10% by weight relative to the total weight of the composition, such as from 2% to 4% by weight relative to the total weight of the composition.

The composition of the present invention may further comprise at least one thickening agent. Non-limiting examples of the at least one thickening agent include those listed on pages 1810 to 1812 of the CTFA International Cosmetic Ingredient Dictionary and Handbook, 8^{th} Ed. (2000). The thickening agent may, for example, be chosen from synthetic thickening agents such as acrylic polymer derivatives. Non-limiting examples of such acrylic polymer derivatives are acrylic acid polymers sold under the tradename CARBOPOL by B. F. Goodrich and acrylamide polymers sold under the tradename SEPIGEL by SEPPIC. The at least one thickening agent, if present, may be present in the inventive composition in an amount ranging up to 20% relative to the total weight of the composition.

The inventive composition may further comprise at least one additive commonly used in cosmetic and/or dermatological compositions. Non-limiting examples of the at least one additive include colorants, hydrating agents, vitamins, antiwrinkle agents, essential fatty acids, sunscreen agents, emollients, preserving agents, neutralizing agents, cosmetic and dermatological active agents, antioxidants, alcohols, plasticizing agents, and waxes. In one embodiment, the at least one additive is not a plasticizing agent. In another embodiment, the at least one additive is not a wax. Another non-limiting example of the least one additive is silicone compounds different from the at least one humectant and from the at least one thickening agent. Non-limiting examples of these silicone compounds include cyclopentasiloxane (e.g., sold as DC 245 Fluid by Dow Corning) and a mixture of cyclopentasiloxane and dimethiconol (e.g., sold as DC 1501 Fluid by Dow Corning). The at least one additive, if present, may be present in the composition in an amount ranging up to 99% of the total weight of the composition.

As previously discussed, the inventive composition further comprises at least 20% water. Water may be present in the composition in an amount ranging up to 95% of the total weight of the composition. For example, in one embodiment, water is present in the aqueous composition in an amount greater than or equal to 20% by weight relative to the total weight of the composition. In another embodiment, water is present in the aqueous composition in an amount greater than or equal to 50% by weight relative to the total weight of the composition, such as greater than or equal to 75% by weight, and further such as greater than or equal to 85% by weight.

The presently claimed composition may, for example, be in the form of a liquid, a lotion, a cream, a paste, a gel, or a solid, and may, further for example, be a pigmented, i.e., colored, composition, such as a foundation, a pre-makeup base, or an unpigmented composition. Further, the inventive composition may be a single emulsion (such as an oil-in-water or water-in-oil emulsion), a multiple emulsion (such as an oil-in-water-in-oil emulsion or a water-in-oil-in-water emulsion), or a rigid or soft gel. In one embodiment, the composition of the invention may be an aqueous composition. In another embodiment, the composition may be an aqueous gel. Aqueous gels have the advantage of being transparent and non-greasy upon application. They are generally stable systems and have a light feel on the skin.

Unless otherwise indicated, all numbers expressing quantities, reaction conditions, and so forth used herein are to be understood as being modified in all instances by the term “about.” Accordingly, unless indicated to the contrary, the numerical parameters set forth in the specification herein and in the attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

### EXAMPLES

### Example 1

Oil Control

The following oil control gel foundation containing 0.5% silica shells, Composition A, was prepared:

| Components of Composition A | Weight Percent |
|---|---|
| Water | 65.9698 |
| Silicones | 12.50 |
| Pigments | 9.0002 |
| Glycerin | 3.00 |
| Thickeners and Preservatives | 4.00 |
| Glycol | 2.00 |
| Fillers | 1.85 |
| Preservative | 0.80 |
| SILICA SHELLS | 0.50 |
| Vitamins | 0.38 |
| Total | 100.00 |

I. Preparation of Composition A

Water, glycerin, preservatives, water soluble vitamins, thickeners and glycol were each individually added to a mixing vessel and mixed until uniform to form a water phase. Silicones and an oil-soluble vitamin were combined to form an oil phase. A pigment grind was prepared and was added to the oil phase with mixing until uniform. The oil phase and the water phase were combined to form an emulsion. The fillers and silica shells were then added to the emulsion and mixed until uniform.

II. Oil Control

Composition A was tested to determine whether a product containing 0.5% silica shells was able to control oil on the skin two hours after application, using blotting paper to determine oil absorption. “Oil control,” as defined above, refers to the reduction or elimination of the amount of oil on the skin. The average temperature for the tests was about 73.4° F. and the average relative humidity was about 66.0%.

A. Test Procedure

Twenty-five panelists having normal-to-oily skin participated in the tests. The test procedure was as follows:
1. The forehead of each panelist was cleaned with an oil-free, alcohol-free makeup remover and allowed to air dry for 5 minutes.
2. An oil-absorbing blotting paper was blotted over the entire forehead of each panelist to determine the baseline amount of oil present, if any. The center and left and right sides of the paper were labeled.
3. 0.2 ml of the composition being tested was applied and spread with a makeup sponge, on the right or left side of the forehead according to a randomization code. The other side of the forehead remained untreated (control).
4. After two hours, the forehead was blotted with a second oil-absorbing blotting paper of the same type used for the baseline test in step 2 (the first blotting paper) the amount of oil present on the forehead in the same manner as in step 2. The center and left and right sides of the paper were labeled.
5. The two blotting papers were then taped onto a piece black paper. The black paper with blotting papers attached was then scanned into a computer using a standard, commercially-available scanner. The computerized image was manipulated (i.e., brightness and contrast adjusted and the image magnified) in order to facilitate observation of the amount of oil on the blotting papers.
6. The amount of oil on each half of a single blotting paper was visually evaluated and rated according to a seven point scale ranging from 0.0 to 3.0 (i.e., by 0.5 increments) where 0.0=no oil absorbed, 1.5=more than a slight amount but less than a moderate amount of oil absorbed, and 3.0=large amount of oil absorbed.
7. A Wilcoxon Signed Rank test was used to compare treated and untreated ratings at baseline and two hours following application of the product being tested. The statistical significance level was set at p-Value≦0.05.

B. Results. Comparison of Scores from the Evaluation of Blotter Paper: Untreated (Control) Sites vs. Treated Sites (Median (25%-75%))

| | Untreated | | |
|---|---|---|---|
| Time Period | (Control) | | |
| (Hours) | Sites | Treated Sites | p-Value |
| Baseline (0) | 0.0 | 0.0 | 1.000 |
| | (0.0-0.0) | (0.0-0.0) | |
| Two Hours | 1.5 | 1.0 | 0.002 |
| | (1.0-2.0) | (0.9-1.5) | |

The sites treated with the inventive composition comprising 0.5% silica shells were statistically significantly less oily than the untreated (control) sites 2 hours after application.

### Example 2

Shine Control and Moisturization

The following six compositions were prepared:
Composition B—Carbopol-based gel make up base containing no silica shells
Composition C—Carbopol-based gel make up base containing 0.6% silica shells
Composition D—Carbopol-based gel make up base containing 1.2% silica shells
Composition E—Sepigel-based clear water make up base containing no silica shells
Composition F—Sepigel-based gel make up base containing 0.6% silica shells
Composition G—Sepigel-based gel make up base containing 1.0% silica shells

All amounts are in percentage by weight.

Table 1: Compositions B-G

TABLE 1
Compositions B-G

| COMPO- | | | | | | |
|---|---|---|---|---|---|---|
| SITION | B | C | D | E | F | G |
| Water | 81.15 | 80.55 | 79.95 | 85.35 | 84.75 | 84.35 |
| Glycerin | 3.00 | 3.00 | 3.00 | 3.00 | 3.00 | 3.00 |
| Preservatives | 0.80 | 0.80 | 0.80 | 0.80 | 0.80 | 0.80 |
| Thickener | 0.90 | 0.90 | 0.90 | 7.00 | 7.00 | 7.00 |
| Solvent | 5.00 | 5.00 | 5.00 | — | — | — |
| Silicones | 3.50 | 3.50 | 3.50 | 3.50 | 3.50 | 3.50 |
| Vitamin | 0.35 | 0.35 | 0.35 | 0.35 | 0.35 | 0.35 |
| Water | 4.70 | 4.70 | 4.70 | — | — | — |
| (deionized) | | | | | | |
| Neutralizing | 0.60 | 0.60 | 0.60 | — | — | — |
| agent | | | | | | |
| SILICA | — | 0.60 | 1.20 | — | 0.60 | 1.00 |
| SHELLS | | | | | | |
| TOTAL | 100.00 | 100.00 | 100.00 | 100.00 | 100.00 | 100.00 |

I. Preparation of Compositions B-G:
Compositions B-D:

Water was added to a kettle and heated. The thickeners, preservatives, vitamins, and solvent were each individually added to the water, and each was mixed until the resultant mixture was fully uniform. The remaining ingredients were added to the kettle and mixed until uniform.

Compositions E-G:

Water, glycerin, vitamins, and preservatives were added to a kettle and mixed until uniform. The thickeners were then added to the kettle and mixed until uniform. The remaining ingredients were added to the kettle and mixed until uniform.

II. Shine Control

Compositions B through G were tested to determine whether each product was able to control shine on the skin two, four and eight hours after application, using a Minolta Multi-Gloss 268 Gloss Meter. “Shine control,” as defined above, refers to the reduction or elimination of the amount of oil on the skin where there is enough excess oil to cause untreated skin to have a “shiny” appearance. The average temperature for the six sets of tests was about 70.2° F. and the average relative humidity was about 58.85%.

A. Test Procedure

The test procedure was as follows:
1. The forehead of each panelist was cleaned with an oil-free, alcohol-free makeup remover and allowed to air dry for 5 minutes.
2. The gloss meter was calibrated and used according to the manufacturer's manual. The geometry mode of 60° was used to obtain the readings. The measurement aperture was placed against the test sites in a vertical position with the illumination originating from the upper part of the unit (closer to the top of the head of the panelist).
3. Baseline measurements were performed on marked sites on each side of the forehead: the “flattest” region of the forehead, which in general was closer to the middle, was chosen as the measurement site. Three measurements were taken at each site and the average of these three measurements on each site was computed for use in data analysis.
4. 0.2 ml of the product being tested was applied and spread with a makeup sponge, on the right or left side of the forehead according to a randomization code. The other side of the forehead remained untreated (control).
5. Gloss meter measurements were taken on the test sites, as in Steps 2 and 3 above, at 2, 4, and 8 hours after product application.
6. The averages of the three readings per site per panelist were analyzed according to standard statistical methods.
   B. Results

Composition B: 30 panelists. No statistically significant differences between the untreated (control) sites and the treated sites at 2, 4, or 8 hours after application. In other words, the product did not control shine at all.

Composition C: 30 panelists. The treated sites were not statistically significantly less shiny than the untreated (control) sites 2, 4, or 8 hours after application.

Composition D: 15 panelists. The treated sites were statistically significantly less shiny than the untreated (control) sites 2 and 8 hours after application. The treated sites were also less shiny than the untreated control sites 4 hours after application, although this difference was not statistically significant. This data suggests that the sample size may not have been large enough to detect a statistically significant change at 4 hours.

Composition E: 30 panelists. No statistically significant differences between the shine of the untreated (control) sites and the treated sites at 2, 4, or 8 hours after application.

Composition F: 30 panelists. The treated sites were not statistically significantly less shiny than the untreated (control) sites 2, 4, or 8 hours after application.

Composition G: 34 panelists. The treated sites were statistically significantly less shiny than the untreated (control) sites 2, 4, and 8 hours after application.

III. Moisturization

Compositions B through G were tested to determine the hydrating efficiency of each product on the skin two, four and eight hours after application using Corneometer CM820 (Courage & Khazaka). As defined above, “moisturizing” refers to hydrating the skin and also retaining moisture in the skin. The average temperature for the six sets of tests was about 70.4° F. and the average relative humidity was about 45.5%.

A. Test Procedure

The test procedure was as follows:
1. A randomization schedule was followed and the initial baseline hydration measurements on all sites (positive control, untreated control and product site) on the inner ulnar forearm of each panelist were taken using the Corneometer CM820.
2. Following the randomization schedule, a site on the ulnar forearm was either left untreated, treated with a positive control or treated with an amount of product being tested equal to about 2 mg/cm². For liquid-like products, a micropipettor was used to draw 10 μl of the composition. For thicker compositions, the appropriate amount was weighed using a microbalance. The product was spread onto the test panelists' forearms using a glass rod.
3. Hydration measurements were taken on the sites, as above, at 2, 4 and 8 hours after product application.
4. The hydration measurements of the treated area were compared with those of the untreated area.
   B. Results

For all six compositions, composition B (comparative, 25 panelists), composition C (comparative, 25 panelists), composition D (inventive, 27 panelists), composition E (comparative, 25 panelists), composition F. (comparative, 27 panelists) and composition G (inventive, 27 panelists), the product was found to statistically significantly hydrate the skin 2, 4, and 8 hours after application compared to the untreated control. The positive control also statistically significantly hydrated the skin 2, 4, and 8 hours after application compared to the untreated control.

Summary of Results from Example 2

| COMPOSITION | B | C | D | E | F | G |
|---|---|---|---|---|---|---|
| Shine Control | NO | NO | YES | NO | NO | YES |
| for 2, 4, 8 hrs | | | | | | |
| Moisturization | YES | YES | YES | YES | YES | YES |
| for 2, 4, 8, hrs | | | | | | |

1. An aqueous composition for the skin comprising:
(a) porous silica particles having an oil absorption capacity of at least four times by weight their own weight;
(b) at least one humectant; and
(c) at least 20% water,
wherein said porous silica particles and said at least one humectant are present in said composition in a combined amount effective to provide moisturization and oil absorption to the skin.

2. A composition according to claim 1, wherein said composition is an aqueous gel.

3. A composition according to claim 1, wherein said porous silica particles are hollow ellipsoids of silica.

4. A composition according to claim 1, wherein said porous silica particles are spherical porous silica particles.

5. A composition according to claim 1, wherein said porous silica particles have an oil absorption capacity of at least five times by weight their own weight.

6. A composition according to claim 1, wherein said porous silica particles have an oil absorption capacity of at least six times by weight their own weight.

7. A composition according to claim 1, wherein said porous silica particles have an oil absorption capacity of at least seven times by weight their own weight.

8. A composition according to claim 1, wherein said porous silica particles are present in said composition in an amount ranging from 0.5% to 5% by weight relative to the total weight of the composition.

9. A composition according to claim 8, wherein said porous silica particles are present in said composition in an amount of 0.6% to 1.2% by weight relative to the total weight of the composition.

10. A composition according to claim 9 wherein said porous silica particles are present in said composition in an amount of 1.0% to 1.2% by weight relative to the total weight of the composition.

11. A composition according to claim 10, wherein said porous silica particles are present in said composition in an amount greater than 0.6% by weight relative to the total weight of the composition.

12. A composition according to claim 1, wherein said at least one humectant is chosen from glycerin, sodium hyaluronate, sodium PCA.

13. A composition according to claim 12, wherein said at least one humectant is glycerin.

14. A composition according to claim 1, wherein said at least one humectant is present in said composition in an amount ranging from 0.001% to 20% relative to the total weight of the composition.

15. A composition according to claim 14, wherein said at least one humectant is present in said composition in an amount ranging from 1% to 10% relative to the total weight of the composition.

16. A composition according to claim 15, wherein said at least one humectant is present in said composition in an amount ranging from 2% to 4% relative to the total weight of the composition.

17. A composition according to claim 1, further comprising at least one thickening agent.

18. A composition according to claim 17, wherein said at least one thickening agent is chosen from acrylic acid polymers and acrylamide polymers.

19. A composition according to claim 17, wherein said at least one thickening agent is present in said composition in an amount ranging from 0% to 20% relative to the total weight of the composition.

20. A composition according to claim 1, wherein said porous silica particles and said at least one humectant are present in said composition in a combined amount effective to provide both moisturization and oil control to the skin.

21. A composition according to claim 1, wherein said porous silica particles and said at least one humectant are present in said composition in a combined amount effective to provide both moisturization and shine control to the skin.

22. A composition according to claim 21, wherein said porous silica particles and said at least one humectant are present in said composition in a combined amount effective to provide both moisturization and shine control to the skin for up to eight hours.

23. A composition according to claim 1, wherein said water is present in an amount greater than 20% by weight relative to the total weight of said composition.

24. A composition according to claim 1, wherein said water is present in an amount greater than or equal to 50% by weight relative to the total weight of said composition.

25. A composition according to claim 1, further comprising at least one additive chosen from silicone compounds different from said at least one humectant and from said at least one thickening agent, colorants, hydrating agents, vitamins, antiwrinkle agents, essential fatty acids, sunscreen agents, emollients, preserving agents, neutralizing agents, cosmetic active agents, dermatological active agents, antioxidants, and alcohols.

26. A composition according to claim 1, wherein said composition is a single emulsion, a multiple emulsion, or a gel.

27. An aqueous composition for the skin comprising:
(a) porous silica particles having an oil absorption capacity of at least four times by weight their own weight;
(b) at least one humectant; and
(c) at least 20% water;
wherein said porous silica particles and said at least one humectant are present in said composition in a combined amount effective to provide moisturization and oil control to the skin.

28. An aqueous composition for the skin comprising:
(a) porous silica particles having an oil absorption capacity of at least four times by weight their own weight;
(b) at least one humectant; and
(c) at least 20% water;
wherein said porous silica particles and said at least one humectant are present in said composition in a combined amount effective to provide moisturization and shine control to the skin.

29. A method for providing moisturization and oil absorption to skin comprising applying to the skin an aqueous composition comprising porous silica particles having an oil absorption capacity of at least four times by weight their own weight, at least one humectant, and at least 20% water, wherein said porous silica particles and said at least one humectant are present in said composition in a combined amount effective to provide moisturization and oil absorption to the skin.

30. A method according to claim 29, wherein said composition is an aqueous gel.

31. A method according to claim 29, wherein said porous silica particles are hollow ellipsoids of silica.

32. A method according to claim 29, wherein said porous silica particles are spherical porous silica particles.

33. A method according to claim 29, wherein said porous silica particles have an oil absorption capacity of at least five times by weight their own weight.

34. A method according to claim 29, wherein said porous silica particles have an oil absorption capacity of at least six times by weight their own weight.

35. A method according to claim 29, wherein said porous silica particles have an oil absorption capacity of at least seven times by weight their own weight.

36. A method according to claim 29, wherein said porous silica particles are present in said composition in an amount ranging from 0.5% to 5% by weight relative to the total weight of the composition.

37. A method according to claim 36, wherein said porous silica particles are present in said composition in an amount of 0.6% to 1.2% by weight relative to the total weight of the composition.

38. A method according to claim 37, wherein said porous silica particles are present in said composition in an amount of 1.0% to 1.2% by weight relative to the total weight of the composition.

39. A method according to claim 29, wherein said porous silica particles are present in said composition in an amount greater than 0.6% by weight relative to the total weight of the composition.

40. A method according to claim 29, wherein said at least one humectant is chosen from glycerin, sodium hyaluronate, sodium PCA.

41. A method according to claim 40, wherein said at least one humectant is glycerin.

42. A method according to claim 29, wherein said at least one humectant is present in said composition in an amount ranging from 0.001 % to 20% relative to the total weight of the composition.

43. A method according to claim 42, wherein said at least one humectant is present in said composition in an amount ranging from 1% to 10% relative to the total weight of the composition.

44. A method according to claim 43, wherein said at least one humectant is present in said composition in an amount ranging from 2% to 4% relative to the total weight of the composition.

45. A method according to claim 29, wherein said composition further comprises at least one thickening agent.

46. A method according to claim 45, wherein said at least one thickening agent is chosen from acrylic acid polymers and acrylamide polymers.

47. A method according to claim 45, wherein said at least one thickening agent is present in said composition in an amount ranging from 0% to 20% relative to the total weight of the composition.

48. A method according to claim 29, wherein, in said composition, said porous silica particles and said at least one humectant are present in said composition in a combined amount effective to provide both moisturization and oil control to the skin.

49. A method according to claim 29, wherein, in said composition, said porous silica particles and said at least one humectant are present in said composition in a combined amount effective to provide both moisturization and shine control to the skin.

50. A method according to claim 49, wherein, in said composition, said porous silica particles and said at least one humectant are present in said composition in a combined amount effective to provide both moisturization and shine control to the skin for up to eight hours.

51. A method according to claim 29, wherein said composition further comprises at least one additive chosen from silicone compounds different from said at least one humectant and from said at least one thickening agent, colorants, hydrating agents, vitamins, antiwrinkle agents, essential fatty acids, sunscreen agents, emollients, preserving agents, neutralizing agents, cosmetic active agents, dermatological active agents, antioxidants, and alcohols.

52. A method according to claim 29, wherein said composition is a single emulsion, a multiple emulsion, or a gel.

53. A method for providing moisturization and oil control to skin comprising applying to the skin a composition comprising porous silica particles, at least one humectant, and at least 20% water, wherein said porous silica particles and said at least one humectant are present in said composition in a combined amount effective to provide moisturization and oil control to the skin.

54. A method for providing moisturization and shine control to skin comprising applying to the skin a composition comprising porous silica particles, at least one humectant, and at least 20% water, wherein said porous silica particles and said at least one humectant are present in said composition in a combined amount effective to provide moisturization and shine control to the skin.
